(19) United States
(12) Patent Application Publication
(10) Pub. No.: US 2013/0066385 A1
(43) Pub. Date: Mar. 14, 2013

(54) CONNECTING ELEMENT REDUCTION INSTRUMENT AND METHODS FOR USING SAME

(71) Applicant: William Brent Benoist, Germantown, TN (US)

(72) Inventor: William Brent Benoist, Germantown, TN (US)

(73) Assignee: Warsaw Orthopedic, Inc., Warsaw, IN (US)

(21) Appl. No.: 13/232,457

(22) Filed: Sep. 14, 2011

(51) Int. Cl. A61B 17/56

(52) U.S. Cl. 606/86 A

(57) ABSTRACT

A reduction instrument is releasably engageable with a bone anchor and structured to reduce a connecting element into a receiving member of the bone anchor. The reduction instrument includes a first member, a second member pivotably coupled to the first member, an offset anchor engaging portion, and a reduction member axially displaceable relative to the first and second members. A passage may be positioned between the first and second members which includes a laterally enlarged portion positioned proximal to the anchor engaging portion. The reduction member may include a guide member movable along a first interior surface of the first member and facing the second member as the reduction member is axially displaced relative to the first and second members. Methods of use are disclosed.

### BACKGROUND

The present invention relates to a surgical instrument and a manner of using the same, and more particularly but not exclusively relates to the reduction of elongate connecting elements or implant components, such as spinal rods, to one or more bone anchors in an orthopedic construct for treatment of a spinal deformity.

The use of surgical instruments to place components in orthopedic constructs has become commonplace. In particular, spinal implant systems frequently include several bone anchors and an interconnecting rod shaped to provide a desired spinal curvature. Typically, the bone anchors are implanted first and the rod is then fixed to the bone anchors in succession. As this procedure progresses, some degree of force may need to be applied to reduce the distance between the rod and the next anchor to be connected to it. Accordingly, various instruments have been described to facilitate such rod reduction. In this arena, the desire persists for improved rod reducing capability. Thus, there is a need for additional contributions in this area of technology.

### SUMMARY

One embodiment of the present invention is directed to a surgical instrument releasably engageable with a bone anchor and structured to reduce a connecting element into a receiving member of the bone anchor. The instrument includes a first member, a second member pivotably coupled to the first member, an offset anchor engaging portion, and a reduction member axially displaceable relative to the first and second members. In one aspect, a passage positioned between the first and second members includes a laterally enlarged portion positioned proximal to the anchor engaging portion. In a further aspect, the reduction member includes a guide member movable along a first interior surface of the first member facing the second member as the reduction member is axially displaced relative to the first and second members. However, in other embodiments, different forms and applications are envisioned.

Another embodiment of the present invention is directed to a reduction instrument that includes a first member extending between a proximal end portion and an opposite distal end portion offset from the proximal end portion and including a first anchor engaging portion, and a second member pivotably coupled to the first member about a pivot axis and extending between a proximal end portion and an opposite distal end portion offset from the proximal end portion and including a second anchor engaging portion. The instrument also includes a reduction member axially displaceable relative to the first and second members. A passage is positioned between the first and second members distal to the pivot axis and includes a first portion positioned between the first and second anchor engaging portions and a second, laterally enlarged portion positioned proximal to the first portion.

Yet another embodiment of the present invention is directed to a reduction instrument that includes a first member extending between a proximal end portion and an opposite distal end portion, and a second member pivotably coupled to the first member and extending between a proximal end portion and an opposite distal end. The instrument also includes a reduction member that is axially displaceable relative to the first and second members and includes an elongate body extending along a longitudinal axis between a proximal end and an opposite distal end and a guide member laterally offset from the longitudinal axis. The guide member is movable along a first interior surface of the first member facing the second member as the reduction member is axially displaced relative to the first and second members.

A further embodiment of the present invention is directed to a method that includes providing a connecting element and an anchor including a bone engaging portion and a receiver member having a receiving portion structured to receive the connecting element. The method also includes providing a reduction instrument engageable with the anchor. The reduction instrument includes a first member extending between a proximal end portion and an opposite distal end portion offset from the proximal end portion; a second member pivotably coupled to the first member and extending between a proximal end portion and an opposite distal end portion offset from the proximal end portion; and a reduction member axially displaceable relative to the first and second members. The reduction member includes a guide portion and an elongate body extending between a proximal end and an opposite distal end. The method also includes engaging the anchor with the reduction instrument, and distally displacing the reduction member relative to the first and second members, the displacing including moving the guide member in opposing slots of the first and second members.

Another embodiment of the present invention is directed to a unique instrument for surgery in a patient. Other embodiments include unique instruments, methods, systems, devices, kits, assemblies, equipment, and/or apparatus involving the reduction of an elongate connecting element.

Further embodiments, forms, features, aspects, benefits, objects and advantages of the present invention shall become apparent from the detailed description and figures provided herewith.

### BRIEF DESCRIPTION OF THE FIGURES

FIG. 1 is a posterior view of a spinal column segment and a spinal implant system attached thereto.

FIG. 2 is a perspective view of a surgical reduction instrument for positioning an elongate connecting element in a desired position relative to an anchor.

FIG. 3 is a front plan view of the reduction instrument illustrated in FIG. 2.

FIG. 4 is a side plan view of the reduction instrument illustrated in FIG. 2.

FIG. 5 is a distal end view of the reduction instrument illustrated in FIG. 2.

FIG. 6 is a front plan view of a first member of the reduction instrument illustrated in FIG. 2.

FIG. 7 is a side plan view of the first member illustrated in FIG. 6.

FIG. 8 is a section view of the first member illustrated in FIG. 6 taken along view line 8-8.

FIG. 9 is a section view of the first member illustrated in FIG. 6 taken along view line 9-9.

FIG. 10 is a front plan view of a second member of the reduction instrument illustrated in FIG. 2.

FIG. 11 is a side plan view of the second member illustrated in FIG. 10.

FIG. 12 is a section view of the second member illustrated in FIG. 10 taken along view line 12-12.

FIG. 13 is a plan view of a reduction member of the reduction instrument illustrated in FIG. 2.

FIG. 14 is a perspective view of the distal end portion of the reduction member illustrated in FIG. 13.

FIG. 15 is a top plan view of the distal end portion illustrated in FIG. 14.

FIG. 16 is a section view of the distal end portion illustrated in FIG. 14.

FIG. 17 is a side plan view of an engaging member of the reduction instrument illustrated in FIG. 2 structured to engage with the reduction member illustrated in FIG. 13.

FIG. 18 is a section view of the engaging member illustrated in FIG. 17 taken along view line 18-18.

FIG. 19 is a front plan view illustrating the use of the instrument of FIG. 2 for reducing an elongate connecting element into a receiving portion of an anchor.

FIG. 20 is another front plan view illustrating the use of the instrument of FIG. 2 for reducing an elongate connecting element into a receiving portion of an anchor.

FIG. 21 is a side plan view illustrating the use of the instrument of FIG. 2 for reducing an elongate connecting element into a receiving portion of an anchor.

FIG. 22 is a perspective view of a surgical reduction instrument according to one embodiment of the present invention.

FIG. 23 is another perspective view of the reduction instrument illustrated in FIG. 22.

FIG. 24 is a plan view of a reduction member of the reduction instrument illustrated in FIG. 22.

FIG. 25 is a proximal end view of the reduction member illustrated in FIG. 24.

FIG. 26 is a distal end view of the reduction member illustrated in FIG. 24.

FIG. 27 is a plan view of the reduction member illustrated in FIG. 24 with the distal end portion of the reduction member attached.

FIG. 28 is a perspective view of the distal end portion of the reduction member illustrated in FIG. 24.

FIG. 29 is a section view of the distal end portion illustrated in FIG. 28.

FIG. 30 is a top plan view of the engaging member of the reduction instrument illustrated in FIG. 22.

FIG. 31 is a perspective view of the engaging member illustrated in FIG. 30.

FIG. 32 is a perspective view of a surgical reduction instrument according to one embodiment of the present invention.

FIG. 33 is a plan view of the reduction instrument illustrated in FIG. 32.

FIG. 34 is another plan view of the reduction instrument illustrated in FIG. 32 with the distal end portion of the reduction member attached.

FIG. 35 is a detailed view of the distal end of the surgical reduction instrument illustrated in FIG. 32.

FIG. 36 is a perspective view of the distal end portion illustrated in FIG. 32.

FIG. 37 is a sectional view of the distal end portion illustrated in FIG. 32.

### DESCRIPTION OF THE ILLUSTRATED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended. Any such alterations and further modifications in the illustrated devices and described methods, and any such further applications of the principles of the invention as illustrated herein are contemplated as would normally occur to one skilled in the art to which the invention relates.

The present invention is generally directed to a surgical instrument releasably engageable with a bone anchor and structured to reduce a connecting element into a receiving member of the bone anchor. The instrument includes a first member, a second member pivotably coupled to the first member, an offset anchor engaging portion, and a reduction member axially displaceable relative to the first and second members. In one aspect, a passage positioned between the first and second members includes a laterally enlarged portion positioned proximal to the anchor engaging portion. In a further aspect, the reduction member includes a guide member movable along a first interior surface of the first member facing the second member as the reduction member is axially displaced relative to the first and second members. In one form, the elongate connecting element is a spinal rod and the instrument is configured to reduce the spinal rod into one or more receiver members of bone anchors engaged with one or more vertebrae in order to provide support and stabilization of the vertebra(e). However, use of the instrument in other surgical procedures and in connection with anatomical locations other than the spinal column is also contemplated.

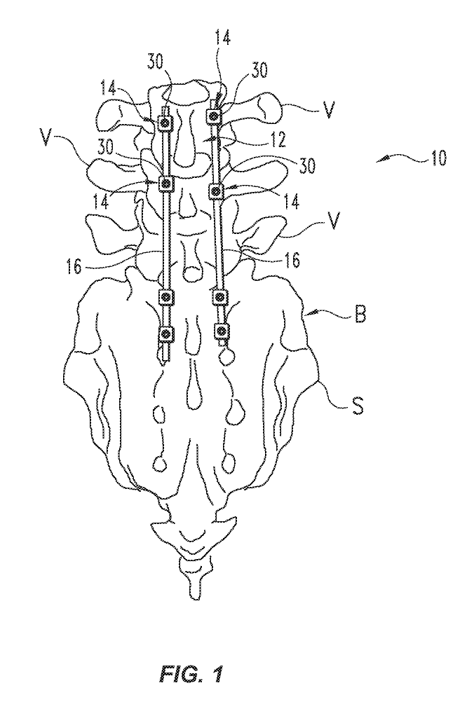

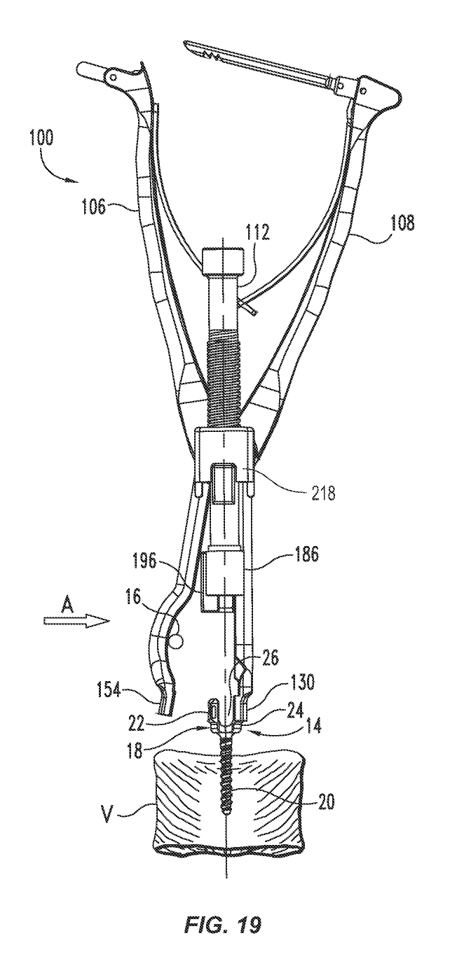

One non-limiting form of a spinal implant system 10 located along a spinal column of a patient and with which the instrument of the present invention may be used is illustrated in FIG. 1. More specifically, in the illustrated form, the implant system 10 is affixed to bones B of the spinal column segment 12 from a posterior approach, although it should be appreciated that implant systems affixed from other surgical approaches to the spine, including anterior, posterior, posterior mid-line, lateral, postero-lateral, and/or antero-lateral approaches, are also contemplated. Bones B include the sacrum S and several vertebrae V. The implant system 10 generally includes several bone anchors 14 and elongate connecting elements 16 structured to selectively interconnect the bone anchors 14. As illustrated in FIGS. 19-21, the bone anchor 14 includes a receiver member 18 configured to receive the connecting element 16 and a distal bone engaging portion 20. In the illustrated embodiment, the bone engaging portion 20 is a bone screw with a threaded shank structured to engage with the bony structure of an underlying vertebra V when the bone anchor 14 is engaged to the spinal column. The receiver member 18 includes a pair of opposing branches 22, 24 defining a channel 26 extending between and opening through opposite first and second sides of the receiver member 18. The branches 22, 24 further define a proximal/distally extending opening that opens through a proximal end of the receiver member 18 to receive a fastening member to secure the connecting element 16 in the channel 26. For example, in one form the fastening member is a set screw structured to engage with internal threading on the branches 22, 24.

In one form, the bone engaging portion 20 is pivotally received in the receiver member 18 through a distal opening thereof, and structured to interact therewith to provide the anchor 14 with multi-axial capabilities that permit either a selected number of positions or an infinite number of positions of the bone engaging portion 20 relative to the receiver member 18. Other forms for the anchor 14 are contemplated, including uni-axial and uni-planar configurations. The bone engaging portion 20 can also be in the form of a spike, staple, hook, fusion device, cannulated screw, fenestrated screw, interbody device, intrabody device, clamp, plate, suture anchor, bolt, pin or other bone engaging configurations. The receiving portion 18 can be in the form of a saddle, yoke, eye-bolt or through-hole, side opening member, bottom opening member, top-opening member, eyelet, or any other structure engageable to the connecting element 16 and the instruments disclosed herein.

In the illustrated embodiment, the connecting element 16 is a rigid spinal rod having a substantially linear configuration between its ends. However, it is contemplated that the connecting element 16 can have a curvature that extends along a constant arc, a varying arc, and/or is compounded between its ends. Additionally, in other forms, the connecting element 16 can include any known configuration for a rod, implant or fastener. It is further contemplated that the connecting element 16 can be non-rigid, elastic and/or super-elastic and in the form of a cable, band, wire, or artificial ligament and may be used in tethering or guiding, and/or in other surgical procedures.

In the implant system 10, the bone anchors 14 are affixed to various locations of the spinal column 12 and interconnected with the connecting elements 16. While the illustrated form of the implant system 10 includes bilaterally positioned connecting elements 16, it should be appreciated that forms in which a single connecting element 16 is positioned on a single side of the spinal column 12 are also possible. Additionally, while not previously discussed, it should be appreciated that the spinal implant system 10 may be used for, but is not limited to, treatment of degenerative spondylolisthesis, fracture, dislocation, scoliosis, kyphosis, spinal tumor and/or a failed previous fusion.

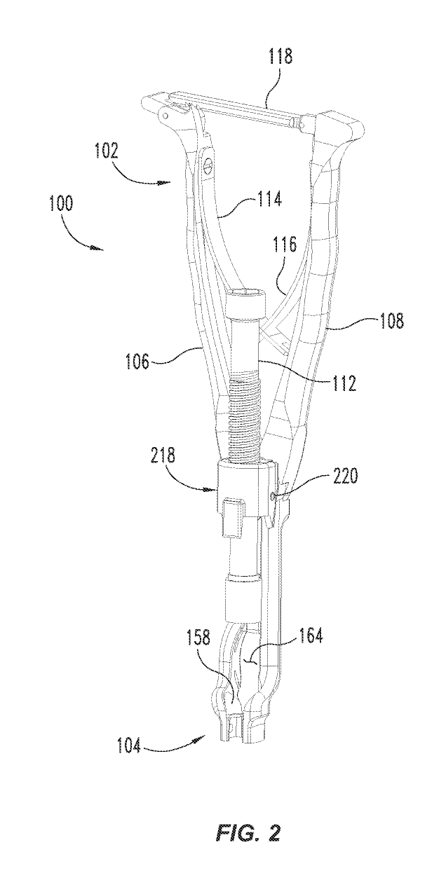

Referring now collectively to FIGS. 2-5, further details of a surgical instrument 100 used to position the connecting element 16 in a desired position relative to one or more of the bone anchors 14 of the spinal implant system 10 will be provided. The instrument 100 generally extends between a proximal end portion 102 configured for engagement and manipulation by a user and a distal end portion 104 configured to releasably engage with one of the bone anchors 14. The instrument 100 also includes a first member 106 and a second member 108 pivotably coupled to the first member 106 about a pivot axis 110, and a reduction member 112 axially displaceable relative to the first and second members 106, 108. The first and second members 106, 108 may be pivotably coupled by a pin that is fixed to one of the members 106, 108 and extends through an aperture in the other of the members 106, 108; a separate pin piece that is secured by swaging, threading, or the like; a hinge; another pivotal coupling of the type used with forceps or scissors, or such different pivotal coupling arrangement as would be known to those skilled in the art.

The proximal end portion 102 of the instrument 100 includes a first spring member 114 coupled to the first member 106 and positioned between the first and second members 106, 108, and a second spring member 116 coupled to the second member 108 and positioned between the first and second members 106, 108. The spring members 114, 116 are generally configured to provide a separation force between the first and second members 106, 108 at the proximal end portion 102 such that the distal end portion 104 is normally open and configured to receive the bone anchor 14. However, the separation force provided by the spring members 114, 116 may be overcome by moving the first and second members 106, 108 toward one another at the proximal end portion 102 in order to engage the bone anchor 14 with the distal end portion 104, further details of which will be provided below. A locking member 118 is coupled to the second member 108 and may be engaged with the first member 106 in order to maintain relative spacing of the first and second members 106, 108 at proximal end portion 102 when distal end portion 104 is engaged with the bone anchor 14.

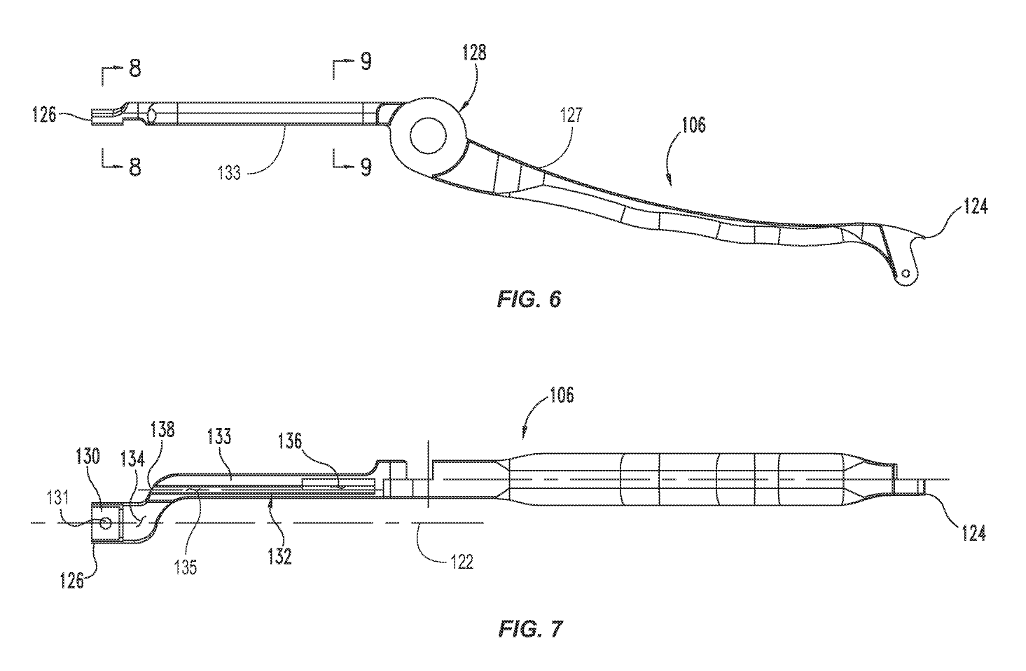

As illustrated in FIG. 4 for example, a first axis 120 generally extends between the first and second members 106, 108 from the proximal end portion 102 toward the distal end portion 104, and a second axis 122 generally extends between the first and second members 106, 108 at the distal end portion 104. The first and second axes 120, 122 generally extend parallel to one another and intersect the pivot axis 110 in an orthogonal or substantially orthogonal relationship, although other variations are possible. Further, the second axis 122 is spaced from the first axis 120 such that the distal end portion 104 is offset from the proximal end portion 102. Further details of this arrangement, amongst others, will be provided below in connection with FIGS. 6-9 and 10-12 where the first and second members 106, 108, respectively, are individually illustrated.

More particularly, turning first to FIGS. 6-9, the first member 106 extends between a proximal end 124 and a distal end 126 that is offset from the proximal end 124 by a curved offset 127, although other variations are possible. A coupling portion 128 is positioned between the proximal and distal ends 124, 126 and configured to engage with a corresponding portion of the second member 108. The distal end 126 includes an anchor engaging portion 130 structured to engage with a first one of the branches 22, 24 of the receiver member 18. A projection 132 extends from the anchor engaging portion 130 and is configured to be positioned in a corresponding receptacle (not shown) on the first one of the branches 22, 24 of the receiver member 18. The first member 106 also includes an arcuately shaped portion 134 that extends about the second axis 122 and is positioned proximal to the anchor engaging portion 130. Arcuately shaped portion 134 generally corresponds in size and shape to a distal end portion of the reduction member 112, further details of which will be provided below.

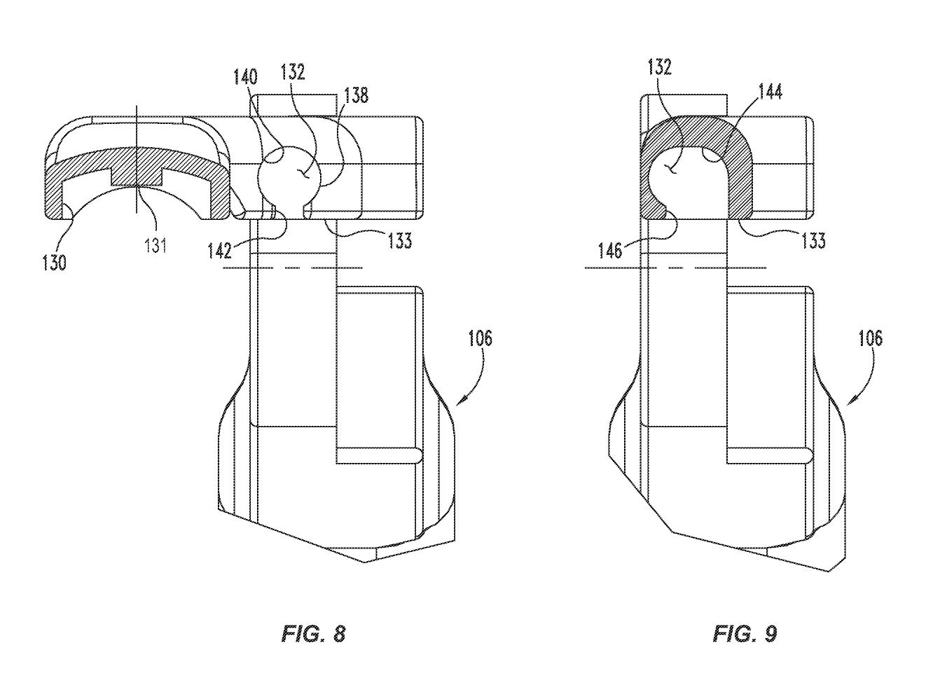

An elongate slot 132 is formed on an inner surface 133 of the first member 106 between the distal end 126 and the coupling portion 128 such that the elongate slot 132 faces a corresponding inner surface of the second member 108 when the first and second members 106, 108 are pivotably coupled. The elongate slot 132 includes a distal portion 135 and a proximal portion 136 that is enlarged relative to the distal portion 135. Additionally, the elongate slot 132 opens through the first member 106 at a distal opening 138 positioned proximal to and offset from the anchor engaging portion 130. As illustrated in FIG. 8, along distal portion 135 the elongate slot 132 includes an enlarged, arcuate portion 140 and a linear portion 142 that communicates with the arcuate portion 140 and opens through the inner surface 133 of the first member 106. As illustrated in FIGS. 7-9, along proximal portion 136 the elongate slot 132 includes an enlarged, partially arcuate portion 144 and a linear portion 146 that communicates with partially arcuate portion 144 and opens through the inner surface 133 of the first member 106. In this arrangement, the proximal portion 136 of the slot 132 is generally configured to allow positioning of a portion of the reduction member 112 into the slot 132 while the distal portion 135 is generally configured to engage with reduction member 112 in a keyed arrangement, further details of which will be provided below.

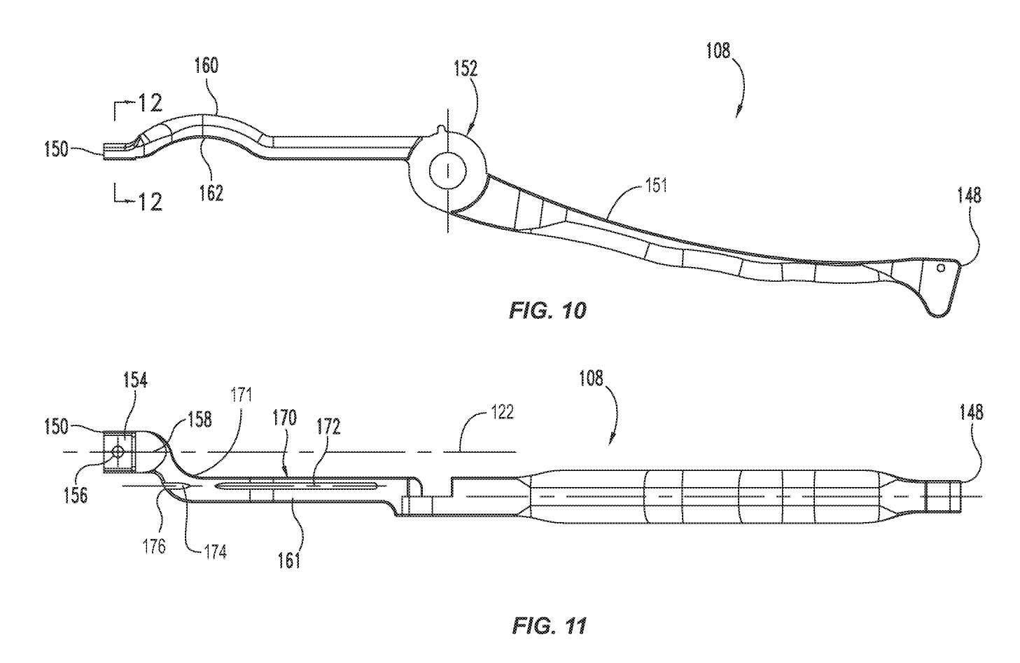

Turning now to FIGS. 10-12, the second member 108 extends between a proximal end 148 and a distal end 150 offset from the proximal end 148 by a curved offset 151, although other variations are possible. A coupling portion 152 is positioned between the proximal and distal ends 148, 150 and configured to engage with the coupling portion 128 of the first member 106. The distal end 150 includes an anchor engaging portion 154 structured to engage with a second one of the branches 22, 24 of the receiver member 18. A projection 156 extends from the anchor engaging portion 154 and is configured to be positioned in a corresponding receptacle (not shown) on the second one of the branches 22, 24 of the receiver member 18. The second member 108 also includes an arcuately shaped portion 158 that extends about the second axis 122 and is positioned proximal to the anchor engaging portion 154. Arcuately shaped portion 158 generally corresponds in size and shape to a distal end portion of the reduction member 112, further details of which will be provided below.

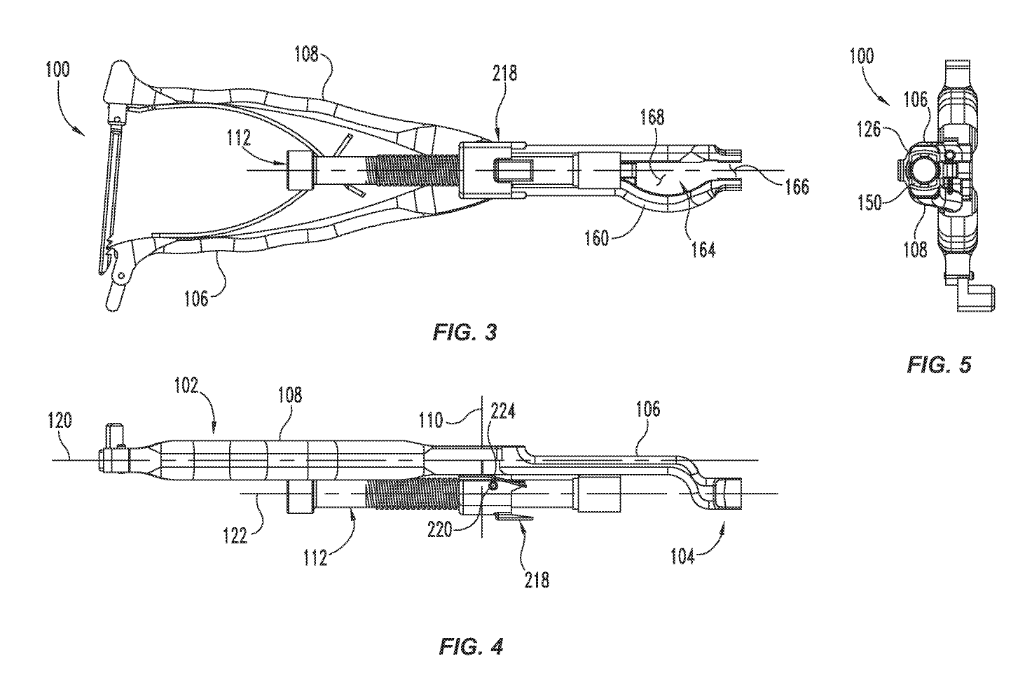

An arcuately curved portion 160 of the second member 108 is positioned between the distal end 150 and the coupling portion 152 and convexly curves away from the first member 106 when the first and second members 106, 108 are pivotably coupled. In this arrangement, an inner surface 161 of the second member 108 that faces the inner surface 133 of the first member 106 when the first and second members 106, 108 are pivotably coupled includes a concave portion 162. As illustrated in FIGS. 2 and 3 for example, when the first and second members 106, 108 are pivotably coupled to one another a passage or space 164 extends between the first and second members 106, 108 distal to the pivot axis 110. The passage 164 is generally configured to receive a portion of the connecting element 16 and includes a first portion 166 positioned between the anchor engaging portions 130, 154 of the first and second members 106, 108 and a second portion 168 that is laterally enlarged relative to the first portion 166 and positioned proximal to the first portion 166. The second, laterally enlarged portion 168 is formed by the arcuately curved portion 160 of the second member 108 and generally facilitates positioning of the connecting element 16 in the passage 164 when it is offset from and misaligned with to the channel 26 of the receiver member 18.

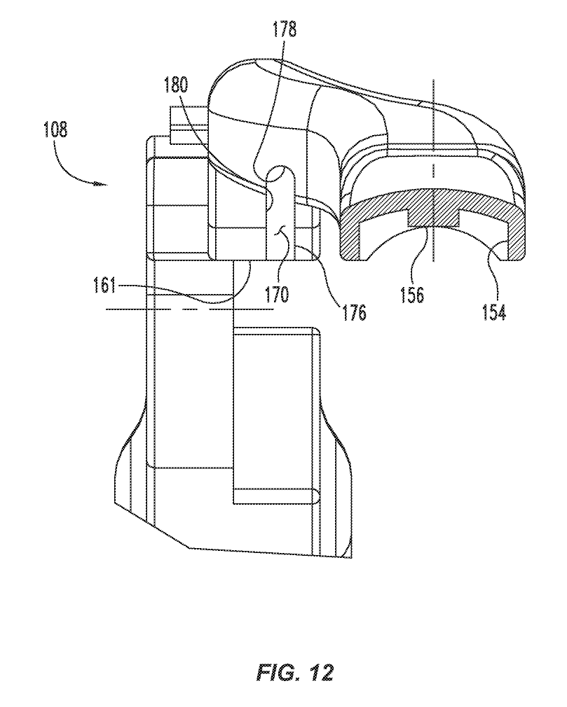

An elongate slot 170 is formed on the inner surface 161 of the second member 108 between the distal end 150 and the coupling portion 152 such that the elongate slot 170 faces the inner surface 133 of the first member 106 when the first and second members 106, 108 are pivotably coupled. The elongate slot 170 is discontinuous due to the arcuately curved portion 160 and includes a proximal portion 172 and a distal portion 174 spaced apart from the proximal portion 172. Additionally, the elongate slot 170 opens through the second member 108 at a distal opening 176 positioned proximal to and offset from the anchor engaging portion 154. As illustrated in FIG. 12, the elongate slot 170 includes an arcuate portion 178 and a linear portion 180 that communicates with the arcuate portion 178 and opens through the inner surface 161 of the second member 108. The elongate slot 170 is generally configured to receive a portion of the reduction member 112, further details of which will be provided below.

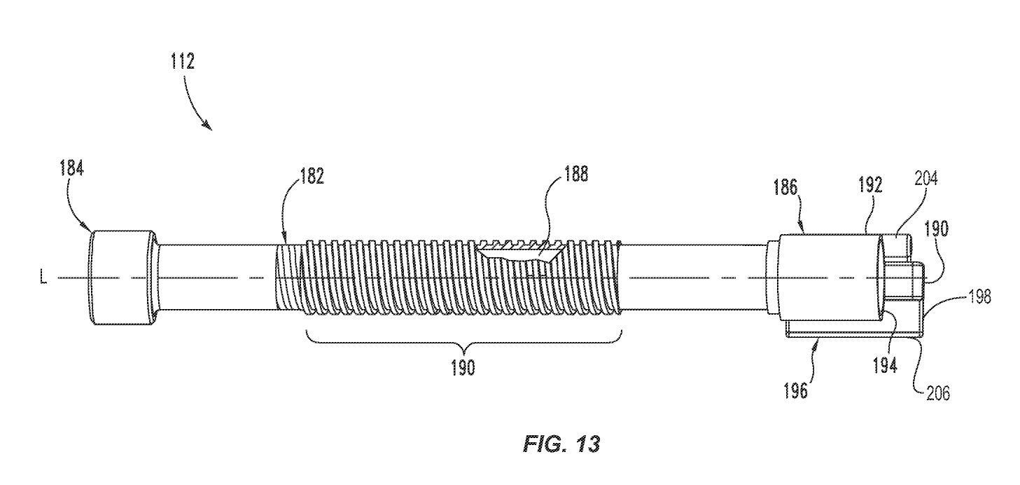

Referring collectively to FIGS. 13-16, further details regarding the reduction member 112 will now be provided. More particularly, in the illustrated form, the reduction member 112 is configured for engaging with the connecting element 16 to reduce the connecting element 16 into the receiver member 18 of the bone anchor 14 and for engaging with the first and second members 106, 108 as it engages with the connecting element 16 in order to increase torsional rigidity of the instrument 100. The reduction member 112 includes an elongated body 182 that extends along a longitudinal axis L between a proximal end portion 184 and an opposite distal end portion 186. Additionally, the reduction member 112 includes an elongate bore 188 extending through the elongated body 182 and opening through the proximal and distal end portions 184, 186 to facilitate passage of a fastening member and related driver through the reduction member 112 in order to secure the connecting element 16 in the channel 26 of the receiver member 18. The elongated body 182 also includes an externally threaded portion 190 that is positioned between the proximal and distal end portions 184, 186 and configured to engage with internal threading of an engaging member, further details of which will be provided below. In one form, the proximal end portion 184 can be provided with an internal receptacle configured to engage with a driving member to facilitate rotation of the reduction member 112 relative to the first and second members 106, 108 when it is engaged with the engaging member.

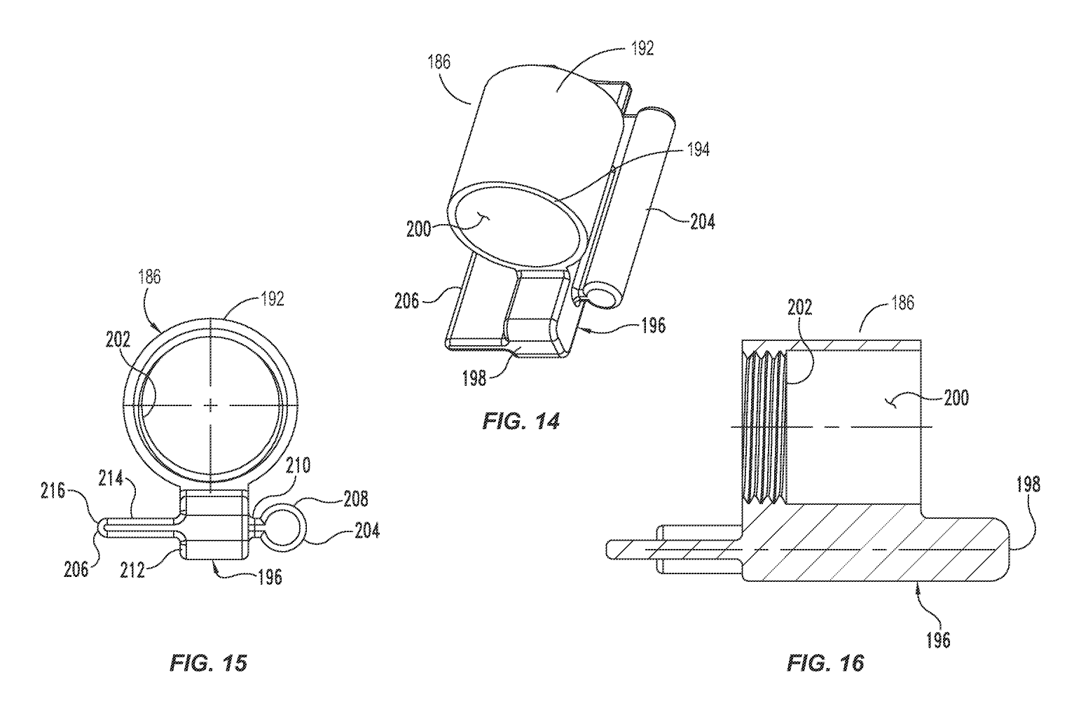

The distal end portion 186 includes an arcuate portion 192 having a distal facing surface 194, and a guide member 196 having a distal end 198 that is positioned distal to the distal facing surface 194. As illustrated in FIGS. 14-16, the distal end portion 186 includes a hollow interior 200 within which is positioned an internally threaded portion 202. The guide member 196 includes oppositely positioned first and second portions 204, 206 which are configured to be received in the elongate slots 132, 170, respectively, of the first and second members 106, 108. The first portion 204 generally includes an arcuate section 208 and a linear section 210 which extends between and couples the arcuate section 208 with an intermediate section 212 of the guide member 196, and the second portion 206 generally includes a linear section 214 extending from the intermediate section 212 to a rounded end portion 216. The elongated body 182 includes an externally threaded portion (not shown) which can be threaded through the internally threaded portion 202 of the distal end portion 186 such that the elongated body 182 is pivotably coupled with the distal end portion 186 and can be rotated relative to the distal end portion 186.

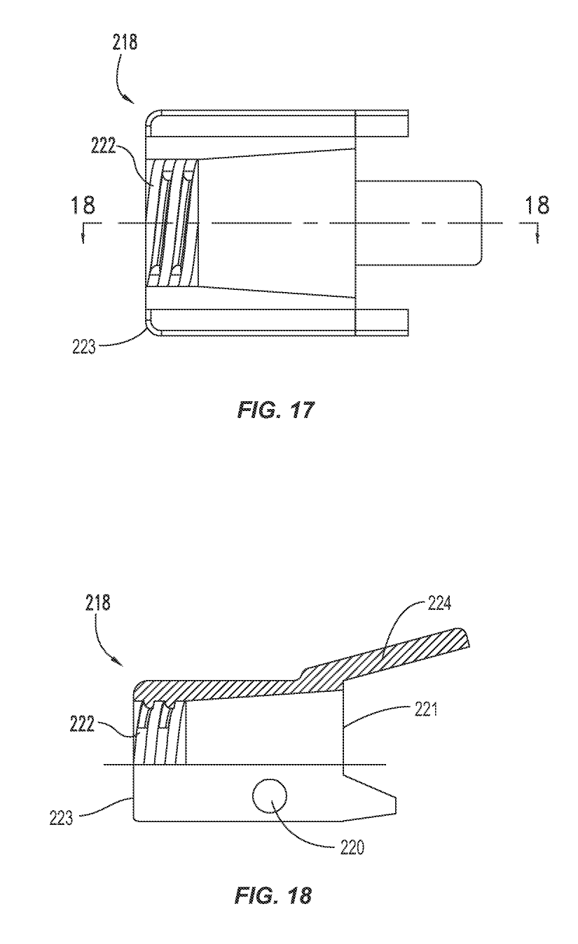

As illustrated in FIGS. 17-18, the instrument 100 also includes an engaging member 218 that is coupled to the coupling member used to pivotably couple the first and second members 106, 108 together. The engaging member 218 is also pivotably movable relative to the first member 106 about a pivot 220. As illustrated in FIGS. 17-18, the engaging member 218 includes an internally threaded portion 222 configured to engage with the externally threaded portion 190 of the reduction member 112. Additionally, a biasing member 224 (shown in FIG. 4) is positioned between the first member 106 and the engaging member 218 such that the proximal end 223 of the engaging member 218 is normally biased toward the first member 106 and the internally threaded portion 222 of the engaging member 218 is engaged with the externally threaded portion 190 of the reduction member 112. When engaged with the engaging member 218, rotation of the reduction member 112 in opposite directions relative to the first and second members 106, 108 distally advances or proximally withdraws the reduction member 112. The distal end 221 of the engaging member 218 may also be depressed toward the first member 106 to displaced the internally threaded portion 222 from engagement with the externally threaded portion 190 of the reduction member 112, in which case the reduction member 112 may be readily displaced relative to the first and second members 106, 108 in both proximal and distal directions without rotation.

When the reduction member 112 is engaged with the engaging member 218, it generally extends in-line with the second axis 122 (as shown in FIG. 4). Similarly, when the reduction member 112 is distally displaced relative to the first and second members 106, 108 (as shown in FIG. 13), the arcuate portion 192 of the distal end portion 186 is received in a receptacle defined by the arcuately shaped portions 134, 158. Additionally, when engaged with the engaging member 218, the reduction member 112 can be proximally withdrawn relative to the first and second members 106, 108 such that the first portion 204 of the guide member 196 is aligned with the proximal portion 136 of the elongate slot 132 of the first member 106. In this arrangement, the first member 106 can be moved relative to the reduction member 112 such that the first portion 204 of the guide member 196 is positioned into the elongate slot 132. Once the first portion 204 of the guide member 196 is positioned in the proximal portion 136 of the elongate slot 132, it can be distally displaced to facilitate positioning of the first portion 204 in the distal portion 135 of the elongate slot 132. In this arrangement, the arcuate section 208 of the first portion 204 of the guide member 196 is positioned in the arcuate portion 140 of the elongate slot 132, and the linear section 210 of the first portion 204 of the guide member 196 is positioned in the linear portion 142 of the elongate slot 132. Similarly, in this arrangement, the guide member 196 and the elongate slot 132 provide a keyed relationship between the first member 106 and the distal end portion 186 of the reduction member 112 such that the distal end portion 186 of the reduction member 112 is not readily displaceable from the elongate slot 132. However, it should be understood that the first portion 204 of the guide member 196 is slidable in the elongate slot 132 to facilitate proximal and distal displacement of the reduction member 112.

As indicated above, the second portion 206 of the guide member 196 is positionable in the elongate slot 170 of the second member 108. More particularly, when the instrument 100 is engaged with the bone anchor 14, the rounded end portion 216 of the second portion 206 is positioned adjacent to the arcuate portion 178 of the elongate slot 170 and the linear section 214 of the second portion 206 is positioned in the linear portion 180 of the elongate slot 170. As the reduction member 112 is proximally and distally displaced relative to the first and second members 106, 108, the second portion 206 of the guide member 196 is slidable in the elongate slot 170. However, in contrast to the arrangement between the guide member 196 and the first member 106 discussed above, it should be understood that the second member 108 can be readily pivoted relative to the first member 106 such that the second portion 206 of the guide member can be positioned into and out of the elongate slot 170 at any number of locations along its length. Additionally, while not previously discussed, it should be appreciated that the engagement of the first portion 204 of the guide member 196 with the first member 106 and of the second portion 206 of the guide member 196 with the second member 108 will increase torsional rigidity of the instrument 100. Similarly, this arrangement enhances the ability to rotate the positioning of the bone anchor 14 and the underlying vertebrae V relative to the connecting element 16 in order to align the bone anchor 14 with the connecting element 16 when the instrument 100 is engaged with the bone anchor 14 and the reduction member 112 is distally displaced to advance the connecting element 16 toward the bone anchor 14.

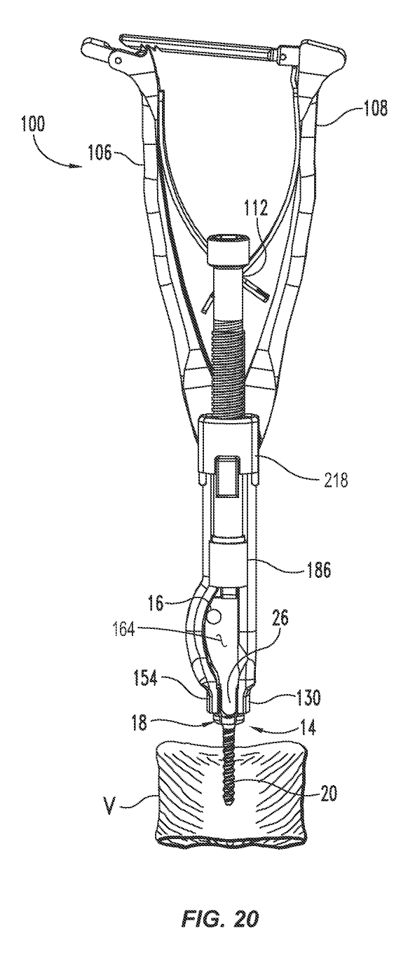

Referring collectively to FIGS. 19-21, further details regarding the operation of the instrument 100 will be provided. More specifically, in FIG. 19, the anchor engaging portion 130 of the first member 106 is engaged with the branch 24 of the receiver member 18 and the anchor engaging portion 154 of the second member 108 is displaced from the receiver member 18. Additionally, the connecting element 16 is laterally offset and misaligned with the channel 26 of the receiver member 18. However, the second member 108 can be pivoted as indicated by directional arrow A in FIG. 19 relative to the first member 106 in order to bring the anchor engaging portion 154 into engagement with the branch 22 of the receiver member 18 and the connecting element 16 into better alignment with the channel 26 of the receiver member 18. Once the connecting element 16 is positioned in the passage 164 between the first and second members 106, 108 as illustrated in FIG. 20, the reduction member 112 can be distally advanced relative to the first and second members 106, 108 into engagement with the connecting element 16. As the reduction member 112 is distally advanced, the connecting element 16 is guided along the concave portion 162 of the inner surface 161 of the second member 108 until it becomes aligned with and positioned in the channel 26 of the receiver member 18. If necessary, the instrument 100 can be rotated in order to rotate the bone anchor 14 and the underlying vertebra V into alignment with the connecting element 16. In view of the foregoing, it should be appreciated that when the connecting element 16 is initially offset from the receiver member 18 as illustrated in FIG. 19 for example, it may be partially moved toward alignment with the receiver member 18 upon engagement of the second member 108 with the receiver member 18, followed by further alignment as it is guided along the second member 108.

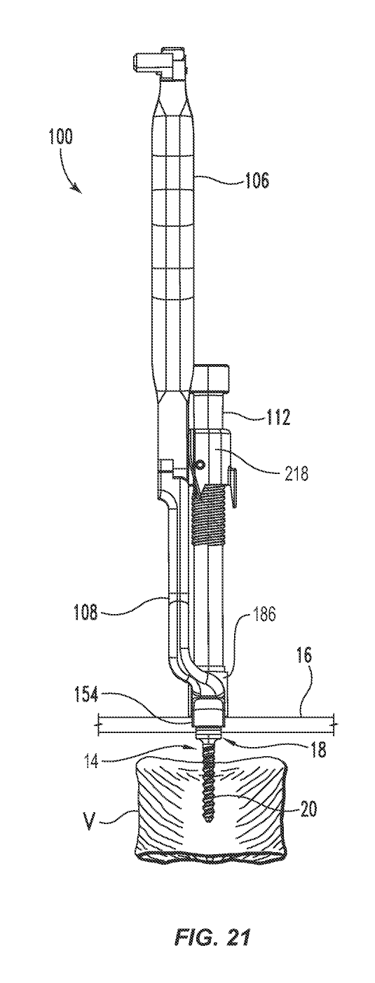

Distal movement of the reduction member 112 can continue until the connecting element 16 is seated in the channel 26 of the receiver member 18 as illustrated in FIG. 21. Additionally, while not previously discussed, it should be appreciated that the connecting element 16 is contacted by the guide member 196 at a location laterally spaced from the receiver member 18 of the bone anchor 14 and the anchor engaging portions 130, 154 of the first and second members 106, 108 (as illustrated in FIG. 19). In this arrangement, the channel 26 and the proximal end of the receiver member 18 remain unobstructed such that a fastening member may be engaged with the receiver member 18 to secure the connecting element 16 in the channel 26. Similarly, once the connecting element 16 is seated in the channel 26, a fastening member and related driver can be advanced through the elongate bore 188 of the reduction member 112 and engaged with the anchor 14 in order to secure the connecting element 16 in the channel 26. Once the connecting element 16 has been properly secured in the channel 26, the reduction member 112 can be proximally displaced relative to the first and second members 106, 108 and the first and second members 106, 108 can be disengaged from the bone anchor 14.

Further details of a surgical instrument 300 used to position a connecting element, such as connecting element 16 in a desired position relative to one or more bone anchors, such as bone anchors 14 of spinal implant system 10 will be provided.

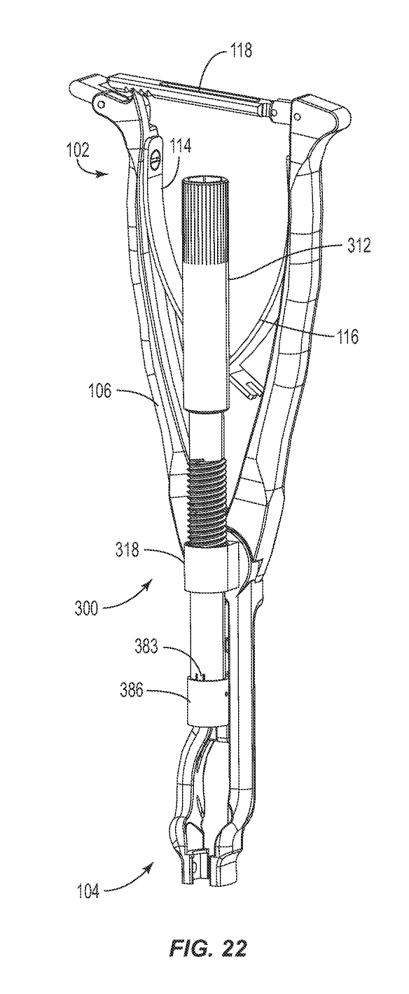

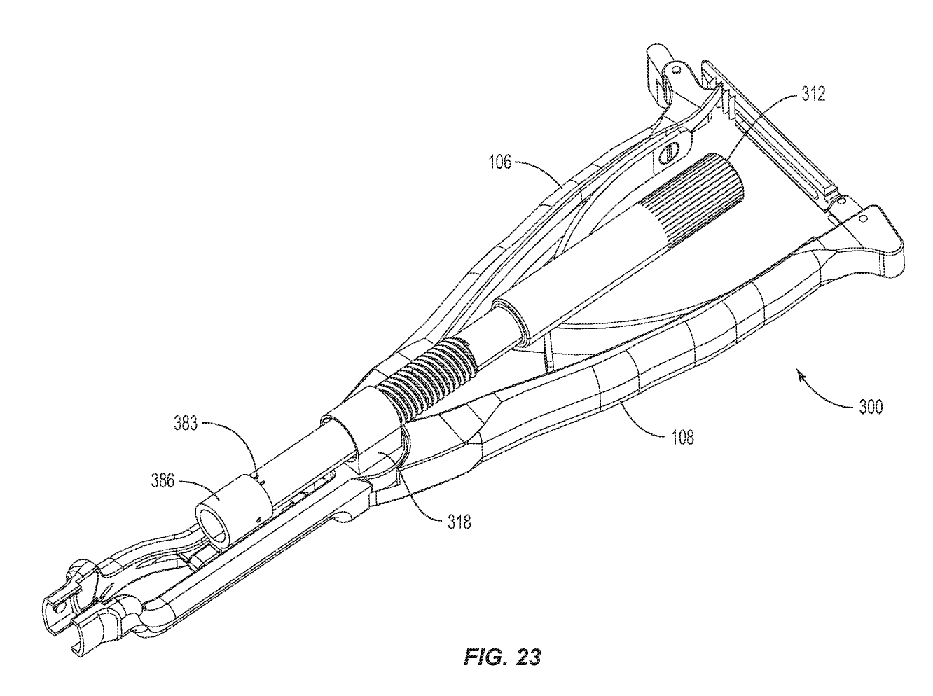

In one embodiment, as shown in FIGS. 22-31, surgical instrument 300 has a configuration similar to that of surgical instrument 100 depicted in FIGS. 2-21, and operates in a similar fashion and can be employed with the surgical methods and procedures described above. Instrument 300 includes a reduction member 312 having an externally threaded portion 390 that is positioned between proximal and distal end portions 384, 386 and configured to engage with an internally threaded portion of an engaging member 318, further details of which will be provided below. When engaged with engaging member 318, rotation of reduction member 312 in opposite directions relative to first and second members 106, 108 distally advances or proximally withdraws reduction member 312, relative to engaging member 318.

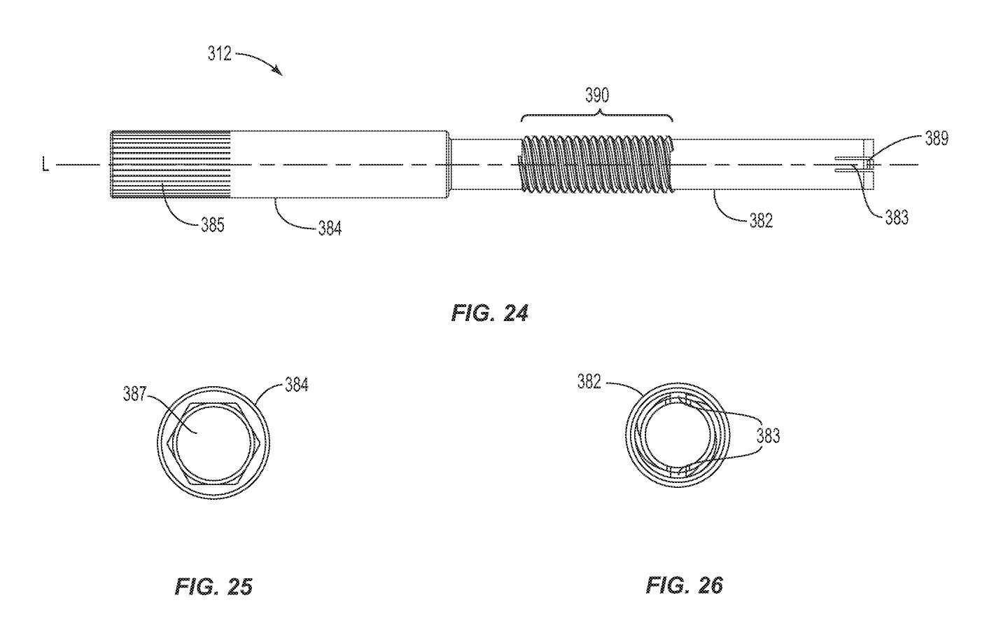

As shown in FIGS. 24-27, reduction member 312 includes an elongated body 382 that extends along a longitudinal axis L between a proximal end portion 384 and an opposite distal end portion 386. Additionally, reduction member 312 includes an elongate bore 388 extending through elongated body 382 and opening through proximal and distal end portions 384, 386 to facilitate passage of a fastening member and related driver through reduction member 312 in order to secure connecting element 16 in channel 26 of receiver member 18. Reduction member 312 has at least one biased tab 383 located at the distal end of elongated body 382 configured to lock a distal end portion onto the distal end of elongated body 382 by engaging a circumferential recess in the distal end portion, further details of which will be discussed below.

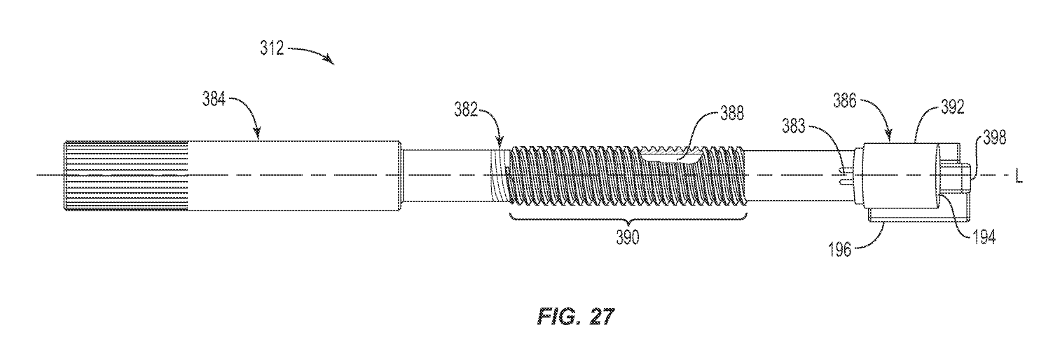

As shown in FIGS. 24 and 27, the diameter of proximal end portion 384 is greater than the diameter of elongated body 382 such proximal end portion 384 may be easily manipulated by a physician to rotate reduction member 312 to distally advance or proximally withdraw reduction member 312, relative to engaging member 318. It is recognized by one of ordinary skill in the art that proximal end portion 384 can be any length, and should be a length in which proximal end portion 384 may be easily manipulated by a physician to rotate reduction member 312 and/or advance a fastening member through elongated bore 388 of reduction member 312 to engage the fastening member with anchor 14 to secure global connecting element 16 in channel 26. Proximal end portion 384 includes a gripping surface 385 that is configured to facilitate rotation of reduction member 312 by providing a stable, grippable surface. Gripping surface 385 comprises a plurality of vertical flutes. It should be understood that proximal end portion 384 may include alternate gripping surfaces that facilitate rotation of reduction member 312.

In one form, proximal end portion 384 includes an internal receptacle 387 configured to engage with a driving member to facilitate rotation of reduction member 312 relative to first and second members 106, 108 described above, when it is engaged with an engaging member. As shown in FIG. 25, internal receptacle 387 is a hexagonal socket which is configured to engage with a hex key. However, internal receptacle 387 can be configured to engage with any type of driving member known in the art sufficient to rotate reduction member 312 to secure connecting element 16 in channel 26 of receiver member 18. Internal receptacle 387 has a hollow center portion which is continuous with elongated bore 388 such that a fastening member may be advanced through elongated bore 388 to secure connecting element 16 in channel 26.

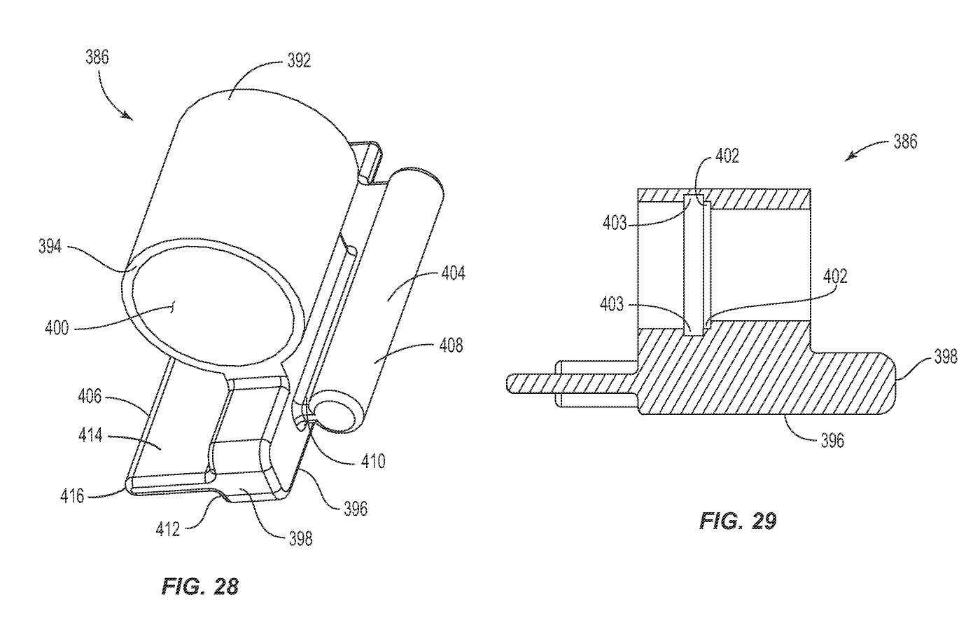

As shown in FIGS. 28-29, distal end portion 386 includes an arcuate portion 392 having a distal facing surface 394, and a guide member 396 having a distal end 398 that is positioned distal to distal facing surface 394. Guide member 396 includes oppositely positioned first and second portions 404, 406 which are configured to be received in elongate slots 132, 170, respectively, of first and second members 106, 108. First portion 404 includes an arcuate section 408 and a linear section 410 which extends between and couples arcuate section 408 with an intermediate section 412 of guide member 396, and second portion 406 includes a linear section 414 extending from intermediate section 412 to a rounded end portion 416.

Distal end portion 386 includes a hollow interior 400 within which is positioned a circumferential recess 402. As discussed above, reduction member 312 has at least one biased tab 383 located at the distal end of elongated body 382 configured to lock distal end portion 386 onto the distal end of elongated body 382 by engaging circumferential recess 402. In particular, tab 383 engages recess 402 by advancing distal end portion 386 over the distal end of elongated body 382 (or vice versa) until tab 383 clicks and locks into place within recess 402. In one particular embodiment of the present invention, the distal end of elongated body 382 includes a plurality of tabs, such as tab 383, located at different locations about the circumference of the distal end of elongated body 382 and each corresponding to a single recess, such as circumferential recess 402, within distal end portion 386.

As shown in FIG. 24, tab 383 includes a locking protrusion 389 which is sized and configured to interface with the corresponding connecting impression 403 in recess 402. Reduction member 312 may be introduced to distal end portion 386 so that tab 383 aligns with recess 402. While advancing reduction member 312, locking protrusion 389 moves longitudinally within hollow interior 400 of distal end portion 385 until locking protrusion 389 of tab 383 reaches connecting impression 403 in recess 402. Distal end portion 386 snaps onto the distal end of reduction member 312. By providing recess 402 circumferentially disposed within distal end portion 386, tab 383 is permitted to rotate within circumferential recess 402 when reduction member 312 is rotated to distally advance or proximally withdraw reduction member 312, relative to engaging member 318 such that distal end portion 385 remains locked onto the distal end of elongated body 382 throughout such rotation of reduction member 312.

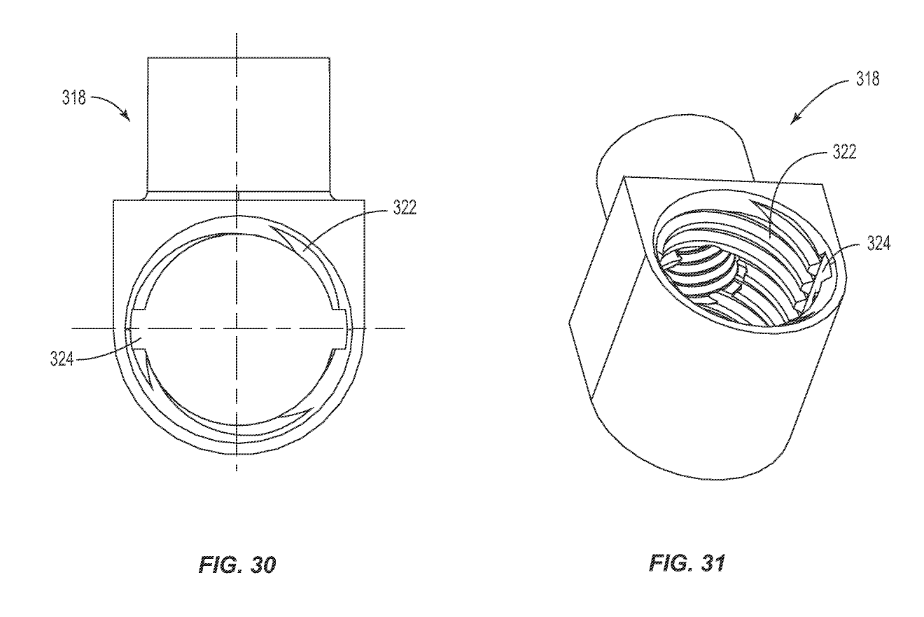

When surgical instrument 300 is assembled, engaging member 318 couples first and second members 106, 108 together. As illustrated in FIGS. 30-31, engaging member 318 includes at least one recess 324 configured such that tab 383 at the distal end of reduction member 312 may pass through the bore in engaging member 318. Engaging member 318 further includes an internally threaded portion 322 configured to engage with externally threaded portion 390 of the reduction member 312. When engaged with engaging member 318, rotation of reduction member 312 in opposite directions relative to first and second members 106, 108 distally advances or proximally withdraws the reduction member 312.

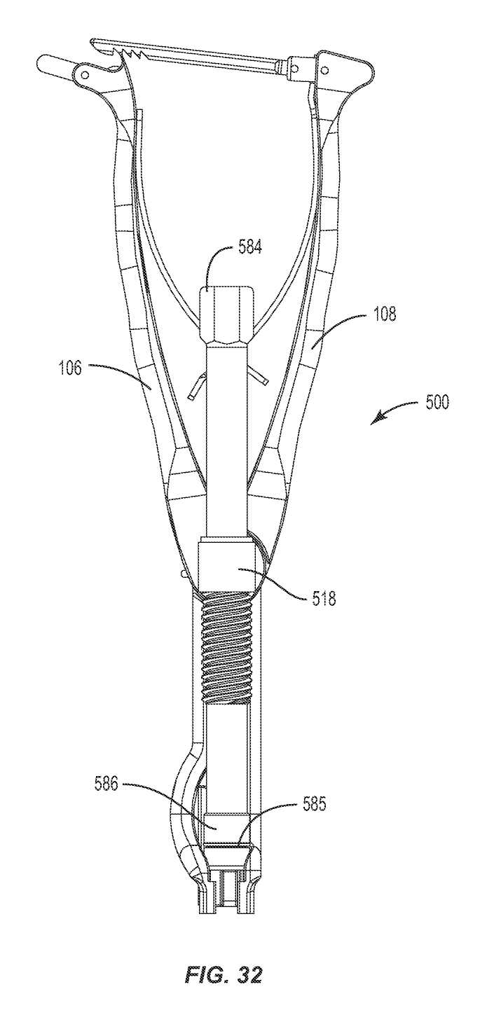

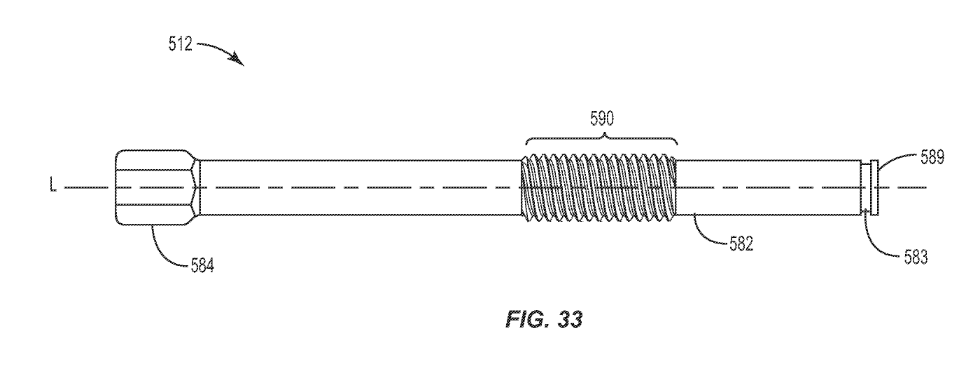

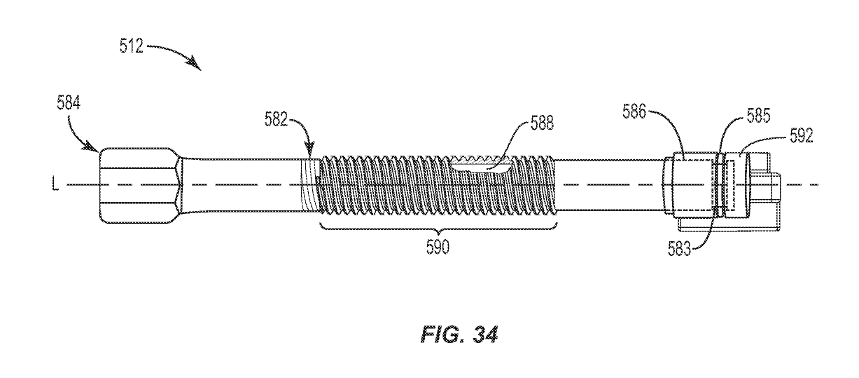

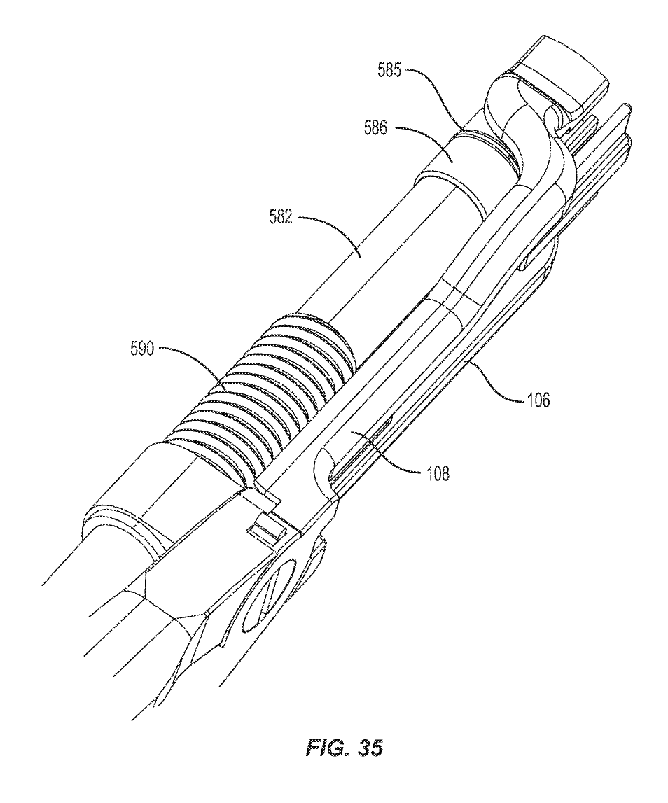

In one embodiment, illustrated in FIGS. 32-37, surgical instrument 500 has a configuration similar to that of surgical instruments 100, 300 depicted in FIGS. 2-31, and operates in a similar fashion and can be employed with the surgical methods and procedures described above. Instrument 500 includes a reduction member 512 having an externally threaded portion 590 that is positioned between proximal and distal end portions 584, 586 and configured to engage with an internally threaded portion of an engaging member, such as an engaging member 518, which has a configuration similar to that of engaging member 318 depicted in FIGS. 22-23 and 30-31. When engaged with engaging member 518, rotation of reduction member 512 in opposite directions relative to first and second members 106, 108 distally advances or proximally withdraws reduction member 512, relative to engaging member 518.

Additionally, reduction member 512 includes an elongate bore 588 extending through an elongated body 582 and opening through proximal and distal end portions 584, 586 to facilitate passage of a fastening member and related driver through reduction member 512 in order to secure connecting element 16 in channel 26 of receiver member 18. Reduction member 512 has a circumferential groove 583 located in a distal end portion 589 of elongated body 582 configured to receive a ring, such as, for example, a snap ring 585 to lock the distal end portion 589 onto the distal end of elongated body 582 by positioning snap ring 585 within groove 583, further details of which will be discussed below.

Proximal end portion 584 has a hexagonal cross-section configured to engage with a driving member having a hexagonal socket to facilitate rotation of reduction member 312 relative to first and second members 106, 108 described above, when it is engaged with engaging member 518. However, proximal end portion 584 may have a cross-section which is triangular, square, pentagonal, hexagonal, or any other polygonal shape and may be configured to engage with a driving member having a socket with a cross-section that corresponds to the cross-section of proximal end portion 584 to rotate reduction member 512 to secure connecting element 16 in channel 26 of receiver member 18. As shown in FIGS. 32-34, the diameter of proximal end portion 584 is greater than the diameter of elongated body 582 such proximal end portion 584 may be easily engaged by a driving member to rotate reduction member 512 to distally advance or proximally withdraw reduction member 512, relative to engaging member 518. However, it is envisioned that proximal end portion 584 may have a diameter that is less than or equal to the diameter of elongated body 582.

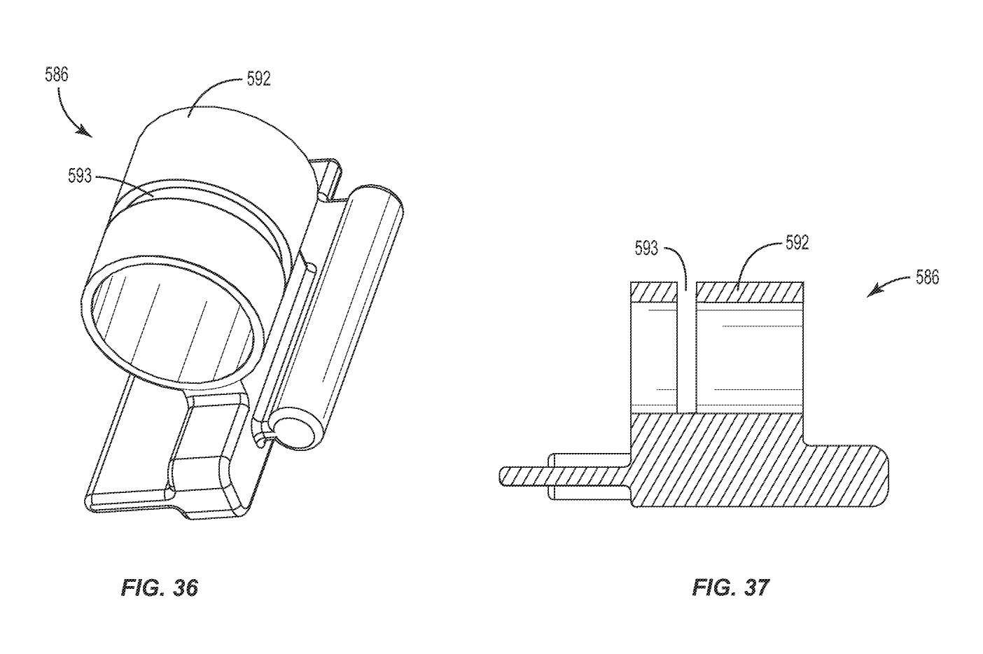

As shown in FIGS. 36-37, distal end portion 586 includes an arcuate portion 592 having a channel 593 disposed therein. As discussed above, reduction member 512 has a circumferential groove 583 located in the distal end of elongated body 582 configured to receive ring 585 to lock distal end portion 586 onto the distal end of elongated body 582 by positioning ring 585 within groove 583. To lock distal end portion 586 onto the distal end of elongated body 582, distal end portion 586 is positioned over the distal end of reduction member 512 such that circumferential groove 583 and channel 593 are aligned. Once circumferential groove 583 and channel 593 are aligned, ring 585 may be inserted into circumferential groove 583 which maintains distal end portion 586 in place, while permitting reduction member 512 to rotate distally to advance or proximally to withdraw reduction member 512, relative to reduction member 512.

Ring 585 may be a semi-flexible ring with open ends which can be snapped into groove 583 on reduction member 512 to permit rotation of reduction member 512 relative to distal end portion 586 and prevent lateral movement of reduction member 512 relative to distal end portion 586. At least one end of ring 585 may include an aperture configured to engage a removal tool to remove ring 585 from groove 583. In one embodiment, ring 585 has a height which is equal to or greater than the combined depth of groove 583 and channel 593 such that at least a portion of ring 585 extends above the outer surface of distal end portion 586. In one embodiment, ring 585 has a height which is less than the combined depth of groove 583 and channel 593 such ring 585 is disposed entirely within groove 583 and channel 593, yet functions to lock distal end portion 586 onto the distal end of reduction member 512.

The instruments, devices, apparatuses, systems and methods described herein also have application with other types of instruments and implants, and may be used in other portions of the body besides the spine. The instruments, devices, apparatuses, systems and methods described herein may also be used in surgical procedures involving animals, or in demonstrations for training, education, marketing, sales and/or advertising purposes. Additionally, the instruments, devices, apparatuses, systems and methods may also be used on or in connection with a non-living subject such as a cadaver, training aid or model, or in connection with testing of surgical systems, surgical procedures, orthopedic devices and/or apparatus.

Any theory, mechanism of operation, proof, or finding stated herein is meant to further enhance understanding of the present invention and is not intended to make the present invention in any way dependent upon such theory, mechanism of operation, proof, or finding. It should be understood that while the use of the word preferable, preferably or preferred in the description above indicates that the feature so described may be more desirable, it nonetheless may not be necessary and embodiments lacking the same may be contemplated as within the scope of the invention, that scope being defined by the claims that follow. In reading the claims it is intended that when words such as “a,” “an,” “at least one,” “at least a portion” are used there is no intention to limit the claim to only one item unless specifically stated to the contrary in the claim. Further, when the language “aleast a portion” and/or “a portion” is used the item may include a portion and/or the entire item unless specifically stated to the contrary.

While the invention has been illustrated and described in detail in the drawings and foregoing description, the same is to be considered as illustrative and not restrictive in character, it being understood that only the selected embodiments have been shown and described and that all changes, modifications and equivalents that come within the spirit of the invention as defined herein or by any of the following claims are desired to be protected.

What is claimed is:

1. A reduction instrument, comprising:
a first member extending between a proximal end portion and an opposite distal end portion offset from said proximal end portion and including a first anchor engaging portion;
a second member pivotably coupled to said first member about a pivot axis and extending between a proximal end portion and an opposite distal end portion offset from said proximal end portion and including a second anchor engaging portion;
a reduction member axially displaceable relative to said first and second members; and
a passage positioned between said first and second members distal to said pivot axis, said passage including a first portion positioned between said first and second anchor engaging portions and a second, laterally enlarged portion positioned proximal to said first portion.

2. The reduction instrument of claim 1, wherein a first axis extends between said first and second members from said proximal end portions transversely through said pivot axis toward said distal end portions, and a second axis is spaced apart from said first axis in a first direction and extends parallel to said first axis between said distal end portions.

3. The reduction instrument of claim 2, wherein said second member includes a portion along said second, laterally enlarged portion of said passage displaced from said first axis in a second direction substantially orthogonal to said first direction.

4. The reduction instrument of claim 2, wherein said reduction member extends along a longitudinal axis between a proximal end portion and an opposite distal end portion, said longitudinal axis being axially aligned with said second axis.

5. The reduction instrument of claim 1, wherein said second member includes a convexly curved portion extending away from said first member along said second, laterally enlarged portion of said passage.

6. The reduction instrument of claim 5, wherein said second, laterally enlarged portion of said passage is defined by a linear inner surface of said first member and a concavely curved inner surface of said second member.

7. The reduction instrument of claim 1, wherein said distal end portion of each of said first and second members includes a concave recess positioned proximal to said anchor engaging portion, said concave recesses cooperating to define a receptacle structured to receive a distal end of said reduction member.

8. The reduction instrument of claim 1, wherein said reduction member includes a guide member and an elongate body extending along a longitudinal axis between a proximal end portion and an opposite distal end portion, said guide member being laterally offset from said longitudinal axis and slidable between said first and second members proximal to said distal end portions as said reduction member is axially displaced relative to said first and second members.

9. The reduction instrument of claim 8, wherein said first member includes a first elongate groove configured to receive a first portion of said guide member in a keyed arrangement and said second member includes a second elongate groove positioned opposite of said first elongate groove and configured to receive a second portion of said guide member.

10. The reduction instrument of claim 8, wherein said guide member includes a distal facing surface positioned distally of a distal facing surface of said elongate body.

11. A reduction instrument, comprising:
a first member extending between a proximal end portion and an opposite distal end portion;
a second member pivotably coupled to said first member and extending between a proximal end portion and an opposite distal end;
a reduction member axially displaceable relative to said first and second members, said reduction member including an elongate body extending along a longitudinal axis between a proximal end and an opposite distal end and a guide member laterally offset from said longitudinal axis; and
wherein said guide member is movable along a first interior surface of said first member facing said second member as said reduction member is axially displaced relative to said first and second members.

12. The reduction instrument of claim 11, wherein said first member includes a first elongate groove on said first interior surface configured to receive a first portion of said guide member in a keyed relationship.

13. The reduction instrument of claim 12, wherein said first portion of said guide member includes a substantially circular portion and a linear portion extending from said substantially circular portion, and said first elongate groove includes an arcuate portion and a linear portion in communication with said arcuate portion.

14. The reduction instrument of claim 13, wherein said first elongate groove includes an enlarged proximal portion configured to receive said first portion of said guide member in non-keyed relationship.

15. The reduction instrument of claim 12, wherein said second member includes a second elongate groove on a second interior surface facing said first member, said second elongate groove being configured to receive a second portion of said guide member.

16. The reduction instrument of claim 15, wherein said first and second members are positionable relative to one another in an anchor engaging configuration and said first portion of said guide member is slidable in said first elongate groove and said second portion of said guide member is slidable in said second elongate groove as said reduction member is axially displaced relative to said first and second members when said first and second members are in said anchor engaging configuration.

17. The reduction instrument of claim 11, further comprising an engaging member pivotably movable relative to said first member, said engaging member including an internally threaded portion configured to engage with an externally threaded portion of said reduction member.

18. A method for reducing a connecting element in a spinal treatment, the method comprising the steps of:
providing a connecting element and an anchor including a bone engaging portion and a receiver member having a receiving portion structured to receive the connecting element;
providing a reduction instrument engageable with the anchor, the reduction instrument including:
a first member extending between a proximal end portion and an opposite distal end portion offset from the proximal end portion;
a second member pivotably coupled to the first member and extending between a proximal end portion and an opposite distal end portion offset from the proximal end portion; and
a reduction member axially displaceable relative to the first and second members, the reduction member including a guide portion and an elongate body extending between a proximal end and an opposite distal end;
engaging the anchor with the reduction instrument; and
distally displacing the reduction member relative to the first and second members, the displacing including moving the guide member in opposing slots of the first and second members.

19. The method of claim 18, wherein distally displacing the reduction member relative to the first and second members includes laterally displacing the connecting element along a portion of the second member into alignment with the receiving portion of the anchor.

20. The method of claim 18, wherein distally displacing the reduction member relative to the first and second members includes engaging the connecting element with the guide member at a location laterally spaced apart from the distal end portions of the first and second members.
